(19) United States
(12) Patent Application Publication
(10) Pub. No.: US 2002/0193812 A1
(43) Pub. Date: Dec. 19, 2002

(54) Hydrogel vaso-occlusive device

(75) Inventors: Tina J. Patel, San Carlos, CA (US); Christopher G.M. Ken, San Mateo, CA (US); Martin S. Dieck, Cupertino, CA (US)

Correspondence Address: BANNER & WITCOFF, 1001 G STREET N W, SUITE 1100, WASHINGTON, DC, 20001, US

(73) Assignee: Concentric Medical, Mountain View, CA

(21) Appl. No.: 10/138,528

(22) Filed: May 6, 2002

Related U.S. Application Data
(60) Provisional application No. 60/288,494, filed on May 4, 2001.

(51) Int. Cl. A61M 29/00

(52) U.S. Cl. 606/151; 606/191

(57) ABSTRACT

Methods and apparatus usable for performing vaso-occlusion at a site of abnormal blood flow in the body. The methods typically comprise treatments for occluding abnormal blood flow in the body, and can be applied to treatment for aneurysms, AVMs, fistulas, ruptured blood vessels and benign or malignant tumors.

### CROSS-REFERENCE TO RELATED APPLICATION

This application claims benefit under 37 CFR §1.78 of provisional application No. 60/288,494, filed May 4, 2001. The full disclosure of the application is incorporated hereby by reference.

### FIELD OF THE INVENTION

The present invention relates to medical devices and methods for vaso-occlusion.

### BACKGROUND OF THE INVENTION

Ruptured blood vessels in the brain cause an acute condition known as hemorrhagic stroke. Ruptures or strokes can occur with a number of vascular abnormalities including arterio venous malformation (AVM), fistulas, aneurysm (a ballooning of the arterial wall), or a burst blood vessel. In addition, abnormal vasculature is generated in the process of tumor growth and tumors including brain tumors and benign or malignant tumors in other parts of the body are highly vascularized entities requiring larger than normal blood flow to sustain the tumor.

Endovascular therapy for vaso-occlusion has included injectable agents, balloon-type occlusive devices, and mechanical vaso-occlusive devices such as metal coils. A description of these agents and devices is included in the background section of U.S. Pat. No. 4,994,069.

Currently, coils for aneurysms and polyvinyl alcohol (PVA) particles for AVMs are FDA approved preventative therapies. Cyanoacrylate glue for AVMs is also proposed and pending approval.

Over 400,000 persons worldwide, and 125,000 persons in the U.S. annually experience some form of hemorrhagic stroke or blood vessel rupture in the brain. A need exists in the medical community and the field of interventional neurology for devices and/or agents that can be used in interventional neurology treatments for strokes and tumors.

Many current embolic devices consist of metal hardware (i.e. platinum, stainless steel, and nitinol) placed into an aneurysm or site of bleeding to fill the space. However, these types of devices are not biodegradable or do not create a biological response to fully occlude the aneurysm, AVM, fistula, ruptured vessel, or benign or malignant tumor vasculature.

### SUMMARY OF THE INVENTION

The present invention includes devices and methods for vaso-occlusion in the body of a patient at a site of abnormal blood flow.

Accordingly, is provided a device for implantation into the vasculature of a patient comprising at least one polymer capable of taking a form that can pass through a delivery device to a site of abnormal blood flow, whereupon the at least one polymer assumes a vaso-occluding shape at the site.

One polymer or two or more polymers can be used in the device. The form that can pass through the delivery device can be selected from the group consisting of a solid or liquid. The solid can be a strip, rod, sheet, roll, tube, ribbon, and coil and the like.

The device can further comprise that when two or more polymers are used the polymers each form a solid and are affixed in a form that can pass through a delivery device and that each polymer differentially responds to an environmental condition.

The environmental condition can be selected from the group consisting of temperature, pH, solvency, pressure, electrical field, and energy source.

The vaso-occluding shape can be selected from the group consisting of a coil, a circle, a half circle, a cone, a twisted sheet, a rod having bends, an amorphous shape, a tangle of filaments, and a helix.

The hydrogel polymer or polymers can be selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic lactic acid (PGLA), a co-polymer of two or more polymers, and a blend of two or more polymers.

The hydrogel polymer or polymers can comprise a natural polymer. The natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, keratin, pectin, elastin, and copolymers and blends of the polymers.

The device can further comprise a bioactive agent reactive at the site of implantation. The bioactive agent can comprises an agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The bioactive agent can be a tissue adhesion factor and the tissue adhesion factor is selected from the group consisting of a fibrin, collagen, albumin, cyanoacrylate, fibrinogen, chitosan, and gelatin-genipin.

The device can further comprise a radio pacifier. The radio pacifier can comprise an agent that provides visibility of the device under X-ray or other imaging technology. The radio pacifier can comprise an agent selected from the group consisting of a contrast media or a metal powder.

A further object of the invention is to provide a method of making the vaso-occlusive device.

Accordingly, is provided, a method of making a vaso-occlusive device for implantation into the vasculature of a patient comprising contacting one or more polymers, copolymers or blended polymers to form a pre-implantation hydrogel polymer, and hydrating the hydrogel polymer either before or during implantation through a delivery device into the patient.

The method can further comprise wherein the device comprises two or more polymers that have a differential sensitivity to an environmental condition, and the two or more polymers contact to form an article for delivery; the method further comprising delivering the article to a site in the patient, wherein the article is exposed to the environmental condition at the site of implantation or during delivery of the article to the site.

Contacting can comprise a process selected from the group consisting of molding, extruding, heating, cooling, shaping, pressurizing, mixing, copolymerizing, affixing, and casting.

The method can further comprise integrating a bioactive agent into one or more polymers for contact and reactivity with material at a site of implantation.

The bioactive agent can comprise an agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The method can further comprise integrating a radio pacifier into one or more polymers for detection of the device in the patient by imaging.

Another object of the invention is to provide a method of treating a patient having abnormal blood flow. Accordingly, is provided a method of treating a patient having abnormal blood flow comprising implanting in the patient at the site of the abnormal blood flow a device comprising one or more polymers capable of being delivered through a delivery device to a site of implantation comprising abnormal blood flow in a patient, whereupon after implantation of the device in the patient the device forms a vaso-occlusive shape.

The polymer or polymers can be selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic lactic acid (PGLA), a co-polymer of two or more polymers, and a blend of two or more polymers.

The method can further comprise integrating a bioactive agent into the vaso-occlusive device for contact and reactivity with material at the site of implantation.

The bioactive agent can be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The method can further comprise integrating a radio pacifier into the vaso-occlusive device for imaging the device after implantation.

### BRIEF DESCRIPTION OF THE DRAWINGS

FIG. 1A shows a delivery device and a strand inside for delivery; FIG. 1B shows the delivered strand in a random arrangement.

FIG. 2A shows a delivery device filed with polymer in a liquid state; FIG. 2B shows an aneurysm filled with polymerized hydrogel in amorphous shapes.

### DETAILED DESCRIPTION OF THE DRAWINGS

The following embodiments and examples are offered by way of illustration and not by way of limitation.

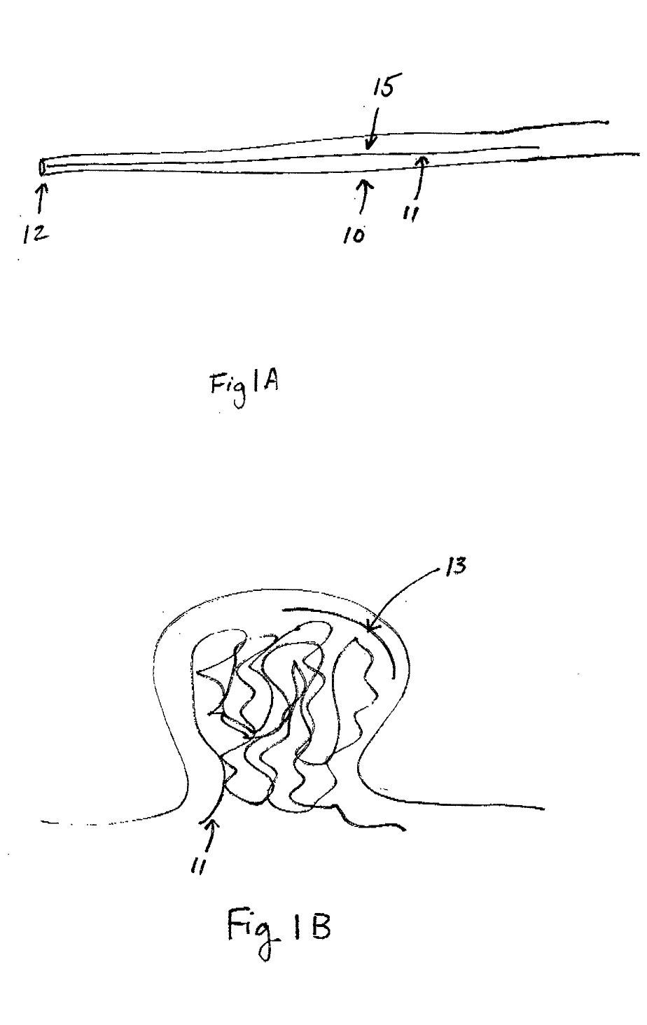

Turning first to the figures, FIG. 1A illustrates a delivery device 10 and a vaso-occlusive device 15 in a pre-implantation shape. In the illustrated embodiment, the vaso-occlusive device 15 includes an article 11 such as a strand positioned within the delivery device 10 for delivery into the body at a location proximate a site of abnormal blood flow. The delivery of the vaso-occlusive device 15 is facilitated at the distal end 12 of the delivery device 10. FIG. 1B shows the delivered strand 11 in a vaso-occlusion shape that includes a random arrangement 13 of the portions of the strand 11.

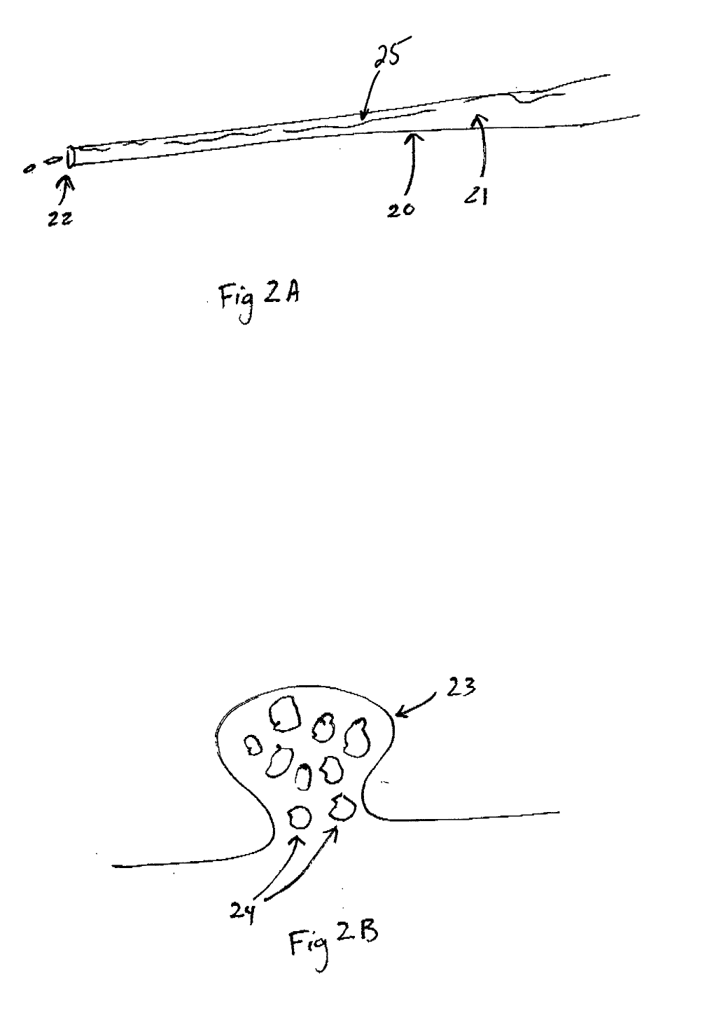

FIG. 2A shows another embodiment of the vaso-occlusion device according to the present invention. In this embodiment, a vaso-occlusion device 25 includes an article 21 formed of a polymer, such as a hydrogel, in a liquid state. In one embodiment, delivery of the device 25 is accomplished by filling a delivery device 20 with the polymer. Upon the placement of the delivery device 20 into a patient's body, the article 21 is delivered into the body from the distal end 22 of the delivery device 20. FIG. 2B shows an aneurysm 23 filled with polymerized hydrogel in amorphous, vaso-occluding shapes 24.

A hydrogel polymer can be used to make either of the vaso-occlusion devices 15, 25 for implantation into a site of abnormal blood flow in a patient. The delivery devices 10, 20 can include a catheter or a pushing device commonly used for delivery of vaso-occluding devices. Thus, the polymer can be a shape or consistency that will allow such delivery, e.g. the polymer can be a liquid or a solid; if a solid the polymer can be e.g. a strip, rod sheet, roll tube, ribbon, coil, or the like. In general the polymer shape of the vaso-occlusion device 15, 25 can be any deliverable shape that can be delivered in a delivery device to a site of abnormal blood flow in a patient. Upon implantation in the patient, as mentioned above, the polymer assumes a vaso-occluding shape and reduces or stops the abnormal blood flow at the site. Two or more hydrogel polymers can be used together either as a co-polymer, or as a blend of polymers to form the material for delivery to the site of abnormal blood flow, and the subsequent vaso-occluding device.

Two or more polymers can be selected for the device where each polymer has a differential sensitivity to an environmental condition (particularly a condition present at the site of implantation, or a condition which can be created in the delivery device, e.g. pH or temperature, or solvency). The two or more polymers are made to contact each other as solids in a shape, and then when the environmental condition differentially affects the contacting polymers, the shape will change because of changes in an affected polymer, thus engineering a vaso-occlusive shape

As shown in the figures, the vaso-occlusive device 15, 25 is intended for implantation into the vasculature of a patient. The implantation site can be any site of abnormal blood flow in the patient, particularly the brain. The abnormal blood flow can be caused by an aneurysm, a ruptured blood vessel, an arterio venous malformation or AVM, a fistula, or a benign or malignant tumor (e.g. cancer or fibroids or the like). Tumors are in part characterized by a highly vascularized state. Otherwise untreatable tumors are particularly contemplated for treatment by implantation of the vaso-occlusive device of the invention, as are uterine fibroids and the like.

The polymer before delivery to the site of abnormal blood flow can have a shape or form that makes it deliverable in the delivery device 10, 20 such as a catheter or pushing device which can be typically used for delivery of embolic or vaso-occlusion devices as shown in the figures and discussed above. Thus, the hydrogel polymer or polymers, or co-polymer, or bended polymers can be in the shape of, for example, but not limited to, a strip, rod, sheet, roll, tube, ribbon, coil, or liquid or semi-liquid. The vaso-occluding shape can be, e.g. a coil that becomes a super coil, coiling back on itself. A strip can fold and twist to form a vaso-occluding rippled and contorted wafer that is no longer flat and two-dimensional. A rod can bend and contort to form a 3-dimensional sphere-like entity taking up space. A roll can unfurl to reveal more layers and can also bend to become more spherical than tubular, and so on. Thus, the vaso-occluding shape of the device 15, 25 that forms upon, soon after or during implantation can be, for example, coil, more complicated a helical coil, a circle, a half circle, a cone, a twisted sheet, a rod of random bends, or a helix, an amorphous shape, or other like or like-acting shapes to name a few possibilities.

In one embodiment of the invention, two or more different hydrogel polymers will typically have a differential sensitivity to an environmental condition. For example, the environmental condition can be provided at the site of implantation, or in a solution or by contacting an agent or environmental condition just before implantation. The environmental condition can be, for example, temperature, pH, solvency, pressure, electrical field, or energy. Exerting a low electrical field, or applying energy to the site can follow implantation of the device to a location in the body. The temperature in the body can provide a differential temperature as compared to outside the body, and upon implantation the device can respond to the differential body temperature. The pH of the environment provided to the device can be neutral after implantation in contrast with slightly acidic or basic before implantation. The solvency differential of the two or more polymers in the device can be facilitated by contacting the device with a semi-liquid environment upon implantation, or a liquid environment during implantation (e.g. if the device is delivered in a liquid stream in a catheter). The environmental condition may be present in the delivery device, for example, or the environmental condition to which two or more polymers are differentially disposed may be present at the site of implantation in the patient.

The change from a first shape to the vaso-occluding shape can be effected in part by the placement of the polymer types in the first shape or form (e.g. a strip or sheet, rod). For example, if one polymer shrinks upon heating, placing that polymer on the underside of a rod, will cause the rod to bend upon exposure to the heat, while the upper part of the rod containing the polymer that does not respond in like manner to the heating condition will be pulled into the secondary shape when the first polymer shrinks. The rod will then form more of a spherical or globular type of vaso-occluding formation.

The hydrogel polymers can be natural or synthetic polymers. The synthetic polymers can be generally any synthetic polymer. The synthetic polymers can be selected from the group consisting of (but not limited to) Polyacrylamide (PAAM), Poly (N-isopropylacrylamine) (PNIPAM), Poly (vinylmethylether), Poly (ethylene oxide), Poly (vinylalcohol), Poly (ethyl (hydroxyethyl) cellulose), Poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic lactic acid (PGLA), co-polymers of two or more polymers, and a blend of two or more polymers. The PGLA disclosed herein can be formed by mixing PGA and PLA in ratios of 99.9:00.1 to 50:50.

The polymer or polymers can be natural polymers, for example selected from the group consisting of (but not limited to) collagen, silk, fibrin, gelatin, hyaluron, cellulose, chitin, dextran, casein, albumin, ovalbumin, heparin sulfate, starch, agar, heparin, alginate, fibronectin, fibrin, elastin, silk-elastin, pectin, copolymers and a blend of two or more polymers.

Conceivably, also, the polymer, polymer pair, copolymer, or polymer blend can be either both or all synthetic polymers, both or all natural polymers, or a mixture of synthetic and natural polymers. Also a single polymer, natural or synthetic may be used to make the device and practice the invention.

The vaso-occlusive device 15, 25 can also comprise a bioactive agent that is reactive at the site of implantation. For example, the bioactive agent may promote maintaining the device at the site of abnormal blood flow, may promote regrowth of a damaged vascular wall, may help to heal the site, may inhibit continued or re-vascularization, may inhibit or regress tumor growth, and such like biological activities at the site of implantation or abnormal blood flow.

Thus, the bioactive agent may be selected from the group consisting of (but not limited to) for example, a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

In the special case, where the bioactive agent is a tissue adhesion factor, for example, to promote keeping the device at the site of implantation, the tissue adhesion factor can be selected from the group consisting of (but not limited to) a fibrin, collagen, albumin, cyanoacrylate, fibrinogen, chitosan, or gelatin-genipin.

The bioactive agent can be integrated into one or both or more polymers useable with the vaso-occlusive device 15, 25 and then releasable as the polymer degrades in the patient. The bioactive agent can be sprayed or coated on the exterior of the vas-occlusion device 15, 25 before its delivery into the patient or during its delivery and then the bioactive agent is released upon contact with material in the body. The application of the bioactive agent to the polymer article can also be accomplished by ion implantation, plasma deposition or vapor deposition, using standard techniques for accomplishing these implantations or depositions of material on articles, known in the art.

The vaso-occlusive device 15, 25 can also comprise a radio pacifier. The radio pacifier can comprise an agent that provides visibility of the device under X-ray or other known imaging technology such as, for example, CT scans, MRIs and flouroscopy. The clear benefit to the device is that it can be monitored and detected once inside the patient. The radio pacifier can comprise, for example, a contrast media or a metal powder, but is not limited to these items. The metal powder can be, for example, titanium, tungsten, gold, bismuth, barium sulfate or tantalum powder. In one embodiment, the radio pacifier includes a gadolinium-based MRI contrast agent. These agents can include, but are not limited to, Gadopentetate, Gadopentetate dimeglumine (Gd DTPA or Magnevist (R)), Gadoteridol (Gd HP-DO3A or ProHance (R)), Gadodiamide (Gd DTPA-BMA or Omniscan (R)), Gadoversetamide (Gd DTPA-BMEA or OptiMARK (R)), Gd-DOTA (Magnevist (R) or Dotarem (R)), Gd-DTPA labeled albumin, and Gd-DTPA labeled dextran.

In an embodiment, the strand 11 is delivered to the surgeon, other practitioner or attendant in pre-cut or pre-formed lengths. In this embodiment, each strand 11 is cut to a predetermined length. For example, the length of the strand 11 of the vaso-occlusive device 15 as it is delivered can be in the range from about 1 mm to about 5 meters. In a preferred embodiment, the pre-cut lengths of the strand 11 of the vaso-occlusive device 15 for delivery to the patient can be in a range from about 1 mm to about 10 mm. In an embodiment, the dimensions of the vas-occlusion device 15 can be from about 0.125 mm to about 12.50 mm, or the outside diameter of objects suitable for passing through a delivery device 10, 20 to a site of abnormal bleeding. The diameter of the vaso-occlusive device 15, 25 once it is delivered and after it has assumed its vaso-occlusion shape (FIGS. 1B, 2B) can be in a range from about 1 mm to about 50 mm.

The invention also includes a method of making the vaso-occlusive device 15, 25 for implantation into the vasculature of a patient comprising contacting one or more polymers, copolymers or blended polymers to form a pre-implantation hydrogel polymer or polymer composition or copolymer, and hydrating the hydrogel either before or during implantation in the patient. Once in the patient the hydrogel assumes a vaso-occluding shape.

The method of making the vaso-occlusive device 15, 25 that comprises two or more polymers that may have a differential sensitivity to an environmental condition, and the two or more polymers can be contacted after each has formed a hydrogel. The method using polymers with differential sensitivities to an environmental condition further comprises delivering the two or more polymers in contact with each other to a site in the patient, wherein the site in the patient or the delivery device comprises an environmental condition to which each polymer is differentially disposed. The environmental condition can be in the hydrant during delivery of the device, or before, when it is being hydrated in anticipation of delivery.

The polymers can be affixed together. For example, if one polymer shrinks upon a raise in temperature above room temperature, the points at which the first polymer is attached to the second non-shrinking polymer, the primary shape of the device bends to form the secondary vaso-occluding shape.

Contacting two or more polymers to form the hydrogel for delivery can comprise a process selected from the group consisting of molding, extruding, heating, cooling, shaping, pressurizing, mixing, copolymerizing, affixing, casting, and blending.

The method of forming a vaso-occlusive device 15, 25 can further comprise integrating a bioactive agent into one or more polymers for contact and reactivity with material at a site of implantation. The bioactive agent can comprise, but is not limited to, for example, an agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

The method of making a vaso-occlusive device can further comprise integrating a radio pacifier into one or more polymers for detection in the patient by imaging.

The invention further provides a method of treating a patient having abnormal blood flow comprising implanting in the patient at the site of the abnormal blood flow the vaso-occlusion device 15, 25 comprising one or more polymers capable of being delivered through the delivery device 10, 20 to an implantation site comprising abnormal blood flow in a patient, whereupon after implantation of the vaso-occlusion device 15, 25 in the patient the device 15, 25 forms its vaso-occlusive shape.

The hydrogel polymer or polymers are selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic lactic acid (PGLA), a co-polymer of two or more polymers, and a blend of two or more polymers.

The vaso-occlusive device 15, 25 and the method of treating a patient can correct or palliate conditions comprising abnormal blood flow such as for example, aneurysm, fistula, ruptured blood vessel, AVM, or benign or malignant tumor. Uterine fibroids are an example of a benign tumor.

Natural and synthetic polymers can be used, as described herein. The device can further comprise a bioactive agent for contact and reactivity with material at the site of implantation also as described herein.

The method can further comprise integrating a bioactive agent into the vaso-occlusion device 15, 25 for contact and reactivity with material at the site of implantation as described above. The bioactive agent can be selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue. The method can further comprise integrating a radio pacifier into the vaso-occlusive device 15, 25 for imaging the device after implantation.

Thermo shrinking polymers that can be used in the vaso-occlusion device 15, 25 are described in Snowden et al, Jul. 15, 1996, Chemistry & Industry features “Some Like It Hot! Thermosensitive Polymers” http://cimond.org/9614/9614111 .html; pages 1-4. Intelligent gels can be used as polymers to form the vaso-occlusive device. Intelligent gels are described in Dagami, Jun. 9, 1997, Chemical & Engineering News pgs. 1-12, C&EN 970609—intelligent gels—http://208.209.231.30/hotartcl/cenear/970609/gels.html.

Ionic-polymer metal composites or IPMCs can be used as the polymers of the vaso-occlusion device 15, 25. IPMCs are described in Shahinpoor, Artificial Muscle Research Institute, School of Engineering and School of Medicine, Univ. of New Mexico, Albuquerque, N. Mex. 87131, “Electro-Mechanics of lono-Elastic Beams as Electrically Controlled Artificial Muscles”.

Bilayer hydrogels can combine two or more polymers for constructing the vaso-occlusive device of the invention. Bilayer hydrogels are described in Calvert and Lui, preprint for SPIE meeting March 1999, “Electrically Stimulated Bilayer Hydrogels as Muscles”.

Other polymers that can be used in combinations to form the vaso-occlusive device 15, 25 are described in Gennes et al, Europhysics Letter vol. 50, no. 4 pp. 513-518, 2000; Thomson, MIT Tech Talk, Wednesday Jun. 12, 1996, vol. 40, no. 33; Perkins, Jan. 17, 2001 “Polymer Films Dissolve to Deliver the Goods” http://www.office.com/global/content/article/printm/0,3232,21846,00.html; pp.1-13. Leary et al, Proc. SPIE Int Soc Opt Eng v3669, p81-86, 1999; Bar-Cohen, Proc SPIE Int Soc Opt Eng v 3669, pp. 57-63, 1999; Caldwell, et al, Mechatronics v. 10; no. 4; pp.499-530, 2000; Baughman et al, Science v.284: n54118, pp. 1340-1344, 1999; Lui and Calvert, Adv. Mater v12, n. 4, pp. 288-291, 2000; Madden et al, Synth Met v. 113: nt, p185-192, 2000; Madden et al, Synth Met v. 105: nl, pp. 61-64, 1999; Hutchinson et al, Synth Met v113: nl, pp. 121-127, 2000; Bar-Cohen et al, Proc SPIE Int Soc Opt Eng v.3041, pp. 697-701, 1997.

U.S. Pat. No. 5,808,012 describes a process that can be used with the present invention by which proteins and other bioactive agents can be incorporated into a polymer during a forming process such as extrusion, molding or casting.

U.S. Pat. No. 6,184,348 describes production of novel polymers using recombinant techniques, and also integration of bioactive agents potentially useful at a site of implantation in the patient. This production can be used with the present invention.

Forming the above-discussed vaso-occlusion device 15, 25 can be accomplished by any number or combination of processes. The two or more above-discussed polymers can be molded together, extruded, heated, cooled, shaped, pressurized, mixed, blended, copolymerized, affixed, and casted, to name a few of the forming processes possible. In addition, a bioactive agent can be integrated into or sprayed on one or both or more polymers so that the bioactive agent can be released or can contact material at the site of implantation. The bioactive agent may also be applied, e.g. by ion implantation, plasma deposition, or vapor deposition. The bioactive agent can be applied to the article as described in U.S. Pat. No. 6,184,348, e.g. by spinning and other means. Contact or release of the bioactive agent provides the opportunity for the factor to act at the site of implantation and provided the intended benefit expected from the biological activity of the factor.

The bioactive agent, as discussed above, can comprise for example a factor selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

All publications, patents and patent applications cited in this specification are herein incorporated by reference as if each individual publication, patent or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A vaso-occlusive device for implantation into the vasculature of a patient comprising:
at least one polymer capable of taking a form that can pass through a delivery device to a site of abnormal blood flow, whereupon the at least one polymer assumes a vaso-occluding shape at the site.

2. A vaso-occlusive device as in claim 1, wherein said at least one polymer includes a single polymer.

3. A vaso-occlusive device as in claim 1, wherein the form that can pass through the delivery device comprises a solid or a liquid.

4. A vaso-occlusive device as in claim 3, wherein the solid is selected from the shapes consisting of a strip, rod, sheet, roll, tube, ribbon, string, and coil.

5. A vaso-occlusive device as in claim 1, wherein said at least one polymer includes at least two polymers, the polymers each form a solid and are affixed in a form that can pass through a delivery device and that each differentially responds to an environmental condition.

6. A vaso-occlusive device as in claim 5, wherein the environmental condition is selected from the group consisting of temperature, pH, solvency, pressure, electrical field, and energy source.

7. A vaso-occlusive device as in claim 1, wherein the vaso-occluding shape is selected from the group consisting of a coil, a circle, a half circle, a cone, a twisted sheet, a rod having bends, an amorphous shape, a tangle of filaments, and a helix.

8. A vaso-occlusive device as in claim 1, wherein the at least one polymer is selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic lactic acid (PGLA), a co-polymer of two or more polymers, and a blend of two or more polymers.

9. A vaso-occlusive device as in claim 1, wherein the at least one polymer comprises a natural polymer.

10. A vaso-occlusive device as in claim 9, wherein the natural polymer can be selected from the group consisting of collagen, silk, fibrin, gelatin, hyaluron, cellulose, chitin, dextran, casein, albumin, ovalbumin, heparin sulfate, starch, agar, heparin, alginate, fibronectin, fibrin, elastin, silk-elastin, pectin, keratin, copolymers thereof, and a blend of polymers.

11. A vaso-occlusive device as in claim 1, further comprising a bioactive agent reactive at the site of implantation.

12. A vaso-occlusive device as in claim 11, wherein the bioactive agent comprises an agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

13. A vaso-occlusive device as in claim 12, wherein the bioactive agent is a tissue adhesion factor and the tissue adhesion factor is selected from the group consisting of a fibrin, collagen, albumin, cyanoacrylate, fibrinogen, chitosan, and gelatin-genipin.

14. A vaso-occlusive device as in claim 1, further comprising a radio pacifier.

15. A vaso-occlusive device as in claim 14, wherein the radio pacifier comprises an agent that provides visibility of the device under an imaging technique.

16. A vaso-occlusive device as in claim 14, wherein the radio pacifier comprises an agent selected from the group consisting of a contrast media or a metal powder.

17. A method of making a vaso-occlusive device for implantation into the vasculature of a patient comprising:
contacting at least one polymer, copolymer or blended polymer to form a pre-implantation hydrogel polymer, and
hydrating the hydrogel polymer either before or during implantation into the patient through a delivery device.

18. A method as in claim 17, wherein the device comprises at least two polymers that have a differential sensitivity to an environmental condition, and said method includes contacting the at least two polymers to form an article for delivery; and delivering the article to a site in the patient; wherein the article is exposed to an environmental condition at the site in the patient or during delivery of the article.

19. A method as in claim 17, wherein contacting comprises a process selected from the group consisting of molding, extruding, heating, cooling, shaping, pressurizing, mixing, copolymerizing, affixing, blending and casting.

20. A method of forming a vaso-occlusive device as in claim 17, further comprising integrating a bioactive agent into said at least one polymer for contact and reactivity with material at a site of implantation.

21. A method as in claim 20, wherein the bioactive agent comprises an agent selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

22. A method as in claim 20, further comprising integrating a radio pacifier into one or more polymers for detection of the device in the patient by imaging.

23. A method of treating a patient having abnormal blood flow comprising:
implanting in the patient at the site of the abnormal blood flow a device comprising at least one polymer capable of being delivered through a delivery device to an implantation site comprising abnormal blood flow in a patient, whereupon after implantation of the device in the patient the device forms a vaso-occlusive shape.

24. A method as in claim 23, wherein the at least one polymer is selected from the group consisting of polyacrylamide (PAAM), poly (N-isopropylacrylamine) (PNIPAM), poly (vinylmethylether), poly (ethylene oxide), poly (vinylalcohol), poly (ethyl (hydroxyethyl) cellulose), poly(2-ethyl oxazoline), Polylactide (PLA), Polyglycolide (PGA), Poly(lactide-co-glycolide) PLGA, Poly(e-caprolactone), Polydiaoxanone, Polyanhydride, Trimethylene carbonate, Poly(β-hydroxybutyrate), Poly(g-ethyl glutamate), Poly(DTH-iminocarbonate), Poly(bisphenol A iminocarbonate), Poly(orthoester) (POE), Polycyanoacrylate (PCA), Polyphosphazene, Polyethyleneoxide (PEO), Polyethylglycol (PEG), Polyacrylacid (PAA), Polyacrylonitrile (PAN), Polyvinylacrylate (PVA), Polyvinylpyrrolidone (PVP), Polyglycolic lactic acid (PGLA), a co-polymer of two or more polymers, and a blend of two or more polymers.

25. A method as in claim 23, further comprising integrating a bioactive agent into the vaso-occlusive device for contact and reactivity with material at the site of implantation.

26. A method as in claim 25, wherein the bioactive agent is selected from the group consisting of a protein factor, a growth factor, an inhibiting factor, an endothelization factor, an extracellular matrix-forming factor, a cell adhesion factor, a tissue adhesion factor, an immunological factor, a healing factor, a vascular endothelial growth factor, a scarring factor, a tumor suppressor, an antigen-binding factor, an anti-cancer factor, a monoclonal antibody, a monoclonal antibody against a growth factor, a drug, a drug producing cell, a cell regeneration factor, a progenitor cell of the same type as vascular tissue, and an a progenitor cell that is histiologically different from vascular tissue.

27. A method as in claim 23, further comprising integrating a radio pacifier into the vaso-occlusive device for imaging the device after implantation.

28. A vaso-occlusive device as in claim 1 wherein said at least one polymer includes a hydrogel polymer.

29. A vaso-occlusive device as in claim 28 wherein said at least one polymer includes at least two hydrogel polymers.

30. A vaso-occlusive device as in claim 1 wherein said at least one polymer includes at least two natural polymers.

31. A vaso-occlusive device as in claim 1 wherein said at least one polymer includes at least one natural polymer and at least one hydrogel polymer.

32. A method as in claim 17 wherein said at least one polymer, copolymer or blended polymer includes at least two hydrogel polymers.

33. A method as in claim 17 wherein said at least one polymer, copolymer or blended polymer includes at least one hydrogel and at least one natural polymer.

34. A method as in claim 17 wherein said at least one polymer, copolymer or blended polymer includes at least two natural polymers.
